(19) United States
(12) Patent Application Publication
(10) Pub. No.: US 2019/0328486 A1
(43) Pub. Date: Oct. 31, 2019

(54) GUIDED ENDODONTIC MICRO-SURGERY (EMS) WITH TREPHINE BURS

(71) Applicant: Government of the United States as represented by the Secretary of the Air Force, Wright-Patterson AFB, OH (US)

(72) Inventors: James A. Wealleans, San Antonio, TX (US); Jarom J. Ray, San Antonio, TX (US)

(73) Assignee: Government of the United States as represented by the Secretary of the Air Force, Wright-Patterson AFB, OH (US)

(21) Appl. No.: 16/396,185

(22) Filed: Apr. 26, 2019

Related U.S. Application Data
(60) Provisional application No. 62/662,931, filed on Apr. 26, 2018.
(60) Provisional application No. 62/662,966, filed on Apr. 26, 2018.

(51) Int. Cl. A61C 5/42; A61B 34/10; A61C 5/44

(52) U.S. Cl. CPC A61C 5/42; A61B 34/10; A61B 2034/107; A61B 2034/105; A61C 5/44

(57) ABSTRACT

Devices and methods for guided endodontic micro-surgery using trephine burs. The device includes a surgical guide comprising a dentate guard and a port extending from the dentate guard. The dentate guard is configured to conform to dentition of a patient proximate to a surgical site. The port has a bore extending therethrough such that a distal end of the bore terminates at the surgical site. The port, and its bore, are configured to receive a trephine bur for the EMS procedure at the surgical site.

### CROSS REFERENCE TO RELATED APPLICATIONS

Pursuant to 37 C.F.R. § 1.78(a)(4), this application claims priority to and benefit of prior filed, co-pending, U.S. Provisional Application Ser. Nos. 62/662,931 and 62/662,966, both filed Apr. 26, 2018. The contents of each application is incorporated herein by reference, each in its entirety.

### RIGHTS OF THE GOVERNMENT

The invention described herein may be manufactured and used by or for the Government of the United States for all governmental purposes without the payment of any royalty.

### FIELD OF THE INVENTION

The present invention relates generally to endodontic surgery and, more particularly, to guided endodontic methods and devices.

### BACKGROUND

Endodontics is a specialized field of dentistry dealing with surgical and therapeutic procedures for protecting tooth pulp or removing tooth pulp from root canals. Tooth pulp is the spongy inner portion of the tooth that contains nerves, arterioles, and venules, as well as lymphatic tissue and fibrous tissue. Removal may be required when a tooth has been injured or diseased since the pulp may die or become necrotic. Conventional endodontic treatments involve preparing an access cavity by removing a substantial part of an occlusal surface of the tooth, removing the coronal pulp, and enlarging the pulp chamber and root canal orifice(s). Often these steps are followed by exploration of the root canal to assess canal length and to extract the radicular pulp. The root canal may then be mechanical shaped with a sequence of instruments. Thereafter the root canal may be cleaned and disinfected by means of irrigation and then filled with a sealing material (usually gutta-percha).

Endodontic micro-surgery (“EMS”) is a surgical procedure utilizing a sophisticated operating microscope and special micro-surgical instruments. Advances in EMS have steadily accumulated over the past 20 years and have resulted in widespread use, greater efficiency, and improved outcomes. For EMS, a 35% increase in weighted, pooled success over antiquated techniques has been reported. EMS achieves desirable outcomes through enhanced visualization, magnification and illumination, micro-surgical instruments, ultrasonic root-end preparations, and use of biocompatible materials. The increased magnification and illumination greatly improves diagnostic capabilities and the precision of surgical procedures.

Nonsurgical root canal treatment and EMS provide viable options for patients dealing with irreversible pulpitis, pulp necrosis, and apical periodontitis. Nevertheless, EMS techniques have higher success rates as compared to traditional approaches.

Certain anatomic considerations may prohibit or limit use of EMS, such as location of neurovascular structures, location toward the posterior dental arch, palatal location in the dental arch, proximity to the maxillary sinus, and areas where bone thickness would prohibit adequate orientation and vision of the root end. For this reason, teeth with prohibitive factors are extracted, with resultant morbidity.

X-ray radiographs are used by most practitioners to assess the extent of disease, understand tooth anatomy, and to plan surgery. However, the two-dimensional nature of radiographs (which are planar projections of 3D objects) can lead to surgical mistakes due to incorrect image interpretation and/or insufficient anatomical information.

The development of computed tomography (“CT”) has enabled clinicians to take radiographic cross-sections and, therefrom, generate 3D reconstructions of maxillofacial features; however, CT necessitates higher radiation exposure to the patient. Cone beam computed tomography (“CBCT”) is an advancement of the CT technology that uses a cone-shaped X-ray beam and a two-dimensional image receptor to generate high-quality, 3D reconstructions with significantly lower radiation exposure. CBCT imaging provides increased visualization of canal morphology, periodontal ligament and bone aberration, root resorption, and appreciation of surrounding anatomic structures.

There remains a need for improvements EMS methods and devices that would enable EMS practice in what would otherwise be considered prohibitive anatomical conditions.

### SUMMARY OF THE INVENTION

The present invention overcomes the foregoing problems and other shortcomings, drawbacks, and challenges of conventional EMS methods, particularly for its use with anatomically difficult presentations. While the invention will be described in connection with certain embodiments, it will be understood that the invention is not limited to these embodiments. To the contrary, this invention includes all alternatives, modifications, and equivalents as may be included within the spirit and scope of the present invention.

Disclosed herein are embodiments of improved methods of endodontic micro-surgery (EMS) that utilize surgical guides that are according to embodiments of the present invention and comprising ports configured to receive a trephine bur and precisely guide the trephine bur to a preselected surgical site on a patient's tooth. By means of computer-aided design of the surgical guide, as well as 3D printing of the guide, the surgical guide may be fabricated such that the trephine bur cuts and removes only desired portions of bone, tooth, soft tissue, and/or root end, while avoiding anatomic structures, such as nerves, sinuses, or blood vessels. In this way, EMS may be carried out in situations that were previously considered too dangerous or too technically challenging, and, in many instances, would have otherwise required tooth extraction.

According to some embodiments of the present invention, a device for endodontic micro-surgery includes a surgical guide having a dentate guard and a port extending from the dentate guard. The dentate guard is configured to conform to dentition of a patient proximate to a surgical site. The port has a bore extending therethrough such that a distal end of the bore terminates at the surgical site. The port, and its bore, are configured to receive a trephine bur for the EMS procedure at the surgical site.

For some aspects of the embodiments, the port is positioned and dimensioned so as to specify the site, angulation, diameter, and depth of a patient specific EMS procedure. Accordingly, other embodiments of the present invention include methods for designing and fabricating the surgical guide, including dimensions and orientation of the port and its bore.

According to another embodiment of the present invention, a device for endodontic micro-surgery includes a surgical guide having a dentate guard and first and second ports extending from the dentate guard. The dentate guard is configured to conform to dentition of a patient proximate to first and second surgical sites. Each of the first and second ports has a bore extending therethrough such that respective distal ends of the bores terminate at respective first and second surgical sites. Each port, and its bore, is configured to receive a trephine bur for the EMS procedure at the respective first and second surgical sites.

Some embodiments of the present invention are direct to a method of performing a patient-specific endodontic micro-surgery procedure by positioning a surgical guide on the dentition of the patient. The surgical guide includes a dentate guard and a port extending from the dentate guard. The dentate guard is configured to conform to dentition of a patient proximate to a surgical site. The port has a bore extending therethrough such that a distal end of the bore terminates at the surgical site. The port, and its bore, are configured to receive a trephine bur for the EMS procedure at the surgical site. After positioning the surgical guide, an osteotomy may be performed through the port.

Other embodiments of the present invention are directed to methods of designing, modeling, and fabricating the surgical guide.

For some embodiments of the present invention, a method of fabricating a three-dimensional surgical guide for a patient includes obtaining a dental model of the patient. At least one parameter of the surgical sight is planned and a virtual model of the surgical guide is prepared. The three-dimensional surgical guide is printed from the virtual model.

Yet other embodiments are directed to a method of fabricating a surgical guide for endodontic micro-surgery by creating a dental model of a surgical guide with a computer-aided design and implant planning software. The dental model is converted to a stereolithographic file, and the surgical guide is printed from the stereolithographic file.

More particularly, according to some embodiments, the method may include the use one or more of CBCT, implant planning software, 3D printed guides, and commercially-available trephine burs to define perforation site, angulation, depth, and diameter of osteotomy, achieving root end resection and biopsy in a single step. The technique is an important breakthrough in surgical endodontics and will enable providers to perform precisely guided surgery in anatomically complex areas for teeth that may have otherwise required extraction.

Moreover, using trephine burs with surgical guides according to embodiments of the present invention yield more successful osteotomies that facilitates autogenous bone graft harvesting.

Some embodiments of the present invention include an access port in the trephine bur port so as to enable sterile water irrigation during trephine bur use.

Yet other embodiments of the present invention dimension the trephine bur port so as to define a depth of trephine bur cutting.

The surgical guides and methods disclosed herein are unique in that they can be used in areas where anatomical complexities render “free-hand” osteotomy and root end resection prohibitive.

While the invention will be described in connection with certain embodiments, it will be understood that the invention is not limited to these embodiments. To the contrary, this invention includes all alternatives, modifications, and equivalents as may be included within the spirit and scope of the present invention.

Additional objects, advantages, and novel features of the invention will be set forth in part in the description which follows, and in part will become apparent to those skilled in the art upon examination of the following or may be learned by practice of the invention. The objects and advantages of the invention may be realized and attained by means of the instrumentalities and combinations particularly pointed out in the appended claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 is a flowchart illustrating a method of fabricating a surgical guide in accordance with an embodiment of the present invention.

FIG. 2 is a diagrammatic view of a surgical guide according to an embodiment of the present invention positioned on the maxillary dentition.

FIG. 3 diagrammatically illustrates a surgical suit that may result from the surgical guide shown in FIG. 2.

FIGS. 4 and 4A are side elevational views of a trephine bur according to an embodiment of the present invention, with FIG. 4A being an enlargement of the portion labeled 4A in FIG. 4.

FIGS. 5 and 6 are a side elevational view and a perspective view of the trephine bur of FIG. 4.

FIGS. 7 and 8 illustrate a method of using a surgical guide according to embodiment of the present invention with a conventional trephine bur (FIG. 7) and the trephine bur of FIG. 4 (FIG. 8).

FIG. 9 is a diagrammatic view of a surgical guide according to an embodiment of the present invention and having a sublingual port for a trephine bur.

FIG. 10 is a diagrammatic view of a surgical guide according to yet another embodiment of the present invention.

FIGS. 11A and 11B are pre-operative photograph and radiograph, respectively, of a patient.

FIG. 11C is a CBCT, coronal image showing a planned trephine path.

FIG. 11D is an image of a 3D model of the planned trephine path.

FIG. 11E is an image of a 3D model of the surgical guide on a digital cast.

FIG. 11F is a photograph of the surgical guide modeled in FIG. 11E.

FIGS. 11G-11J are sequential photographs of the surgical procedure.

FIGS. 11K and 11L are postoperative radiograph and photograph, respectively, of the patient.

FIGS. 11M-11O are sequential postoperative photographs to illustrate healing of the surgical site of the patient.

FIG. 12A is a pre-operative radiograph of a patient.

FIG. 12B is a CBCT axial image showing the fused DF/palatal root and isthmus tooth #14 of the patient.

FIG. 12C is an image of a 3D model of the planned trephine path.

FIG. 12D is an image of a 3D model of the surgical guide on a digital cast.

FIG. 12E is a diagrammatic view of the surgical model in FIG. 12D.

FIGS. 12F-12I are sequential photographs of the surgical procedure.

FIG. 12J is photograph of the extracted core with undebrided DF canal and isthmus.

FIG. 12K is a photograph of the retrograde fill of the canal.

FIGS. 12L-12N are postoperative photograph, radiograph, and photograph respectively, of the patient.

FIGS. 120 and 12P are sequential postoperative photographs to illustrate healing of the surgical site of the patient.

FIG. 13A is a pre-operative radiograph of a patient.

FIG. 13B is a CBCT, coronal image showing a planned trephine path.

FIG. 13C is an image of a 3D model of the planned trephine path.

FIG. 13D is a photograph of the surgical guide modeled in FIG. 13C.

FIGS. 13E and 13F are sequential photographs of the surgical procedure.

FIGS. 13G and 13H are postoperative radiograph and photograph, respectively, of the patient.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION

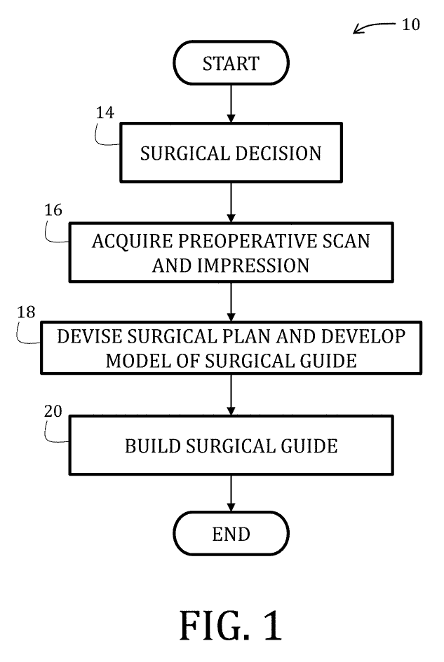

Referring now to the figures, and in particular to FIGS. 1-3, an EMS procedure 10 utilizing a surgical guide 12 according to an embodiment of the present invention is shown. At start, a surgical plan is made (Block 14) and may generally include identifying an area of concern for a particular patient, a number of surgical sites, type of surgical procedure, and so forth. Specifically, but not necessarily, the planning stage may incorporate preoperative scans (such as a cone bean computed tomography, 3-dimensional intraoral scanner, digitized x-ray, and so forth) of a patient, dental impressions, computer models derived from the scans and/or impressions, or combinations thereof (Block 16). According to some embodiments, it may be preferable to use CBCT Digital Imaging and Communications in Medicine (“DICOM”) files that may be converted into stereolithography (“STL”) files for production of a model of the surgical guide, which is also in accordance with the Academy of Oral and Maxillofacial Radiology recommends for presurgical assessments of implant sites.

After evaluating images, impressions, models, or combinations thereof, surgical plan may be determined (Block 18), wherein such details may include location, angulation, depth, and so forth. If desired, suitable surgical planning materials (such as surgical planning software) may be used in determining details of the surgical plan and to development of a model of the surgical guide 12, which is described in greater detail below. The surgical plan may include developing a model for the surgical guide 12 to be fabricated and used in a surgical procedure. Fabrication of the surgical guide 12 may then follow (Block 20) and may include, for example, molding, 3D printing, or other conventional means of manufacturing using the materials described below. It is during the design process that all parameters of angulation and depth of osteotomy may be defined in view of the anatomy of the patient at the surgical site.

One exemplary embodiment may include the use of an 80 mm×80 mm CBCT scan to produce a DICOM file from an impression of the dental arch; however, any scan volume from CBCT or conventional CT scan may be utilized for surgical guide design. The impression may also be subsequently scanned by a benchtop 3D scanner, producing an STL file which is uploaded along with the DICOM file from the CBCT into surgical guide planning software such as Mimics or Bluesky Bio.

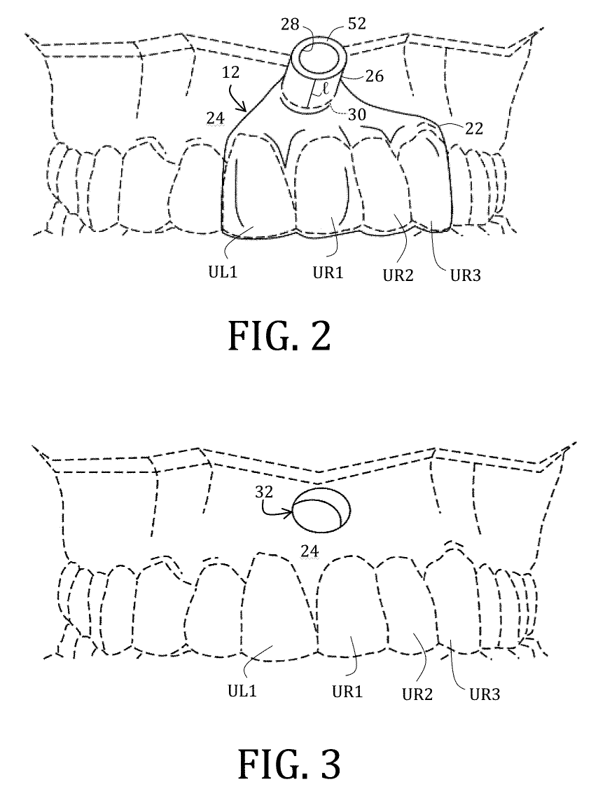

With specific reference now to FIG. 2, the surgical guide 12 according to this particular embodiment of the present invention includes a dentate guard 22 configured to conform to a patient's dental anatomy so as to stabilize surgical guide during the EMS procedure. The dentate guard 22, as shown, conforms to incisors (UR2, UR1, UL1), a canine (UR3) and associated gingiva 24; however, it would be understood by the skilled artisan that other embodiments are not so limited, such as will be shown in greater detail according to other embodiments, below.

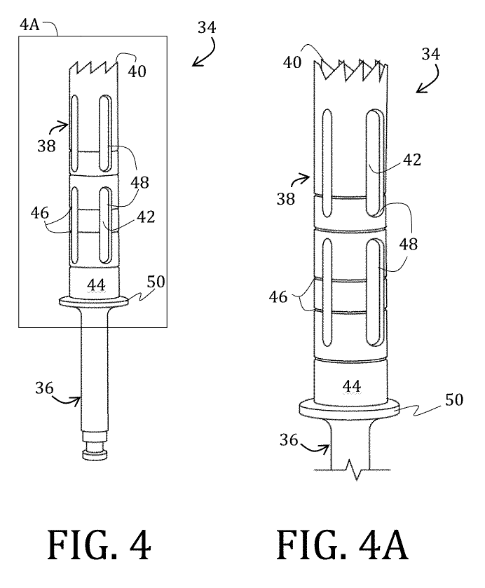

The surgical guide 12 further includes a surgical port 26 having a bore 28 extending therethrough, wherein a distal end 30 of the port 26 aligns to a surgical site 32 for the patient. A diameter of the bore 28 may be configured to receive a trephine bur 34, an embodiment of which according to the present invention is shown in FIG. 4. Alternatively, commercially-available trephines may be used, such as those from 3i, LLC (Palm Beach Gardens, Fla.). Alternatively still, other devices may also be used. More specifically, the diameter of the bore 28 may be determined or selected, at least in part, to accommodate and stabilize a particular trephine bur 34 to be used during the EMS procedure. The trephine bur size may be determined by such factors as root-end width, adjacent anatomical structures, considerations for visualization, surgeon's preference, or combinations thereof. In some embodiments, the diameter of the bore 28 of the port 26 may range from about 2 mm to about 8 mm, with varying diameters to include from about 3.0 mm to about 8.0 mm, from about 4.5 mm to about 7.0 mm, from about 5.0 mm to about 6.5 mm, or from about 5.5 mm to about 6.0 mm.

The port 26 may be positioned and dimensioned, with respect to the dentate guard 22, so as to specify at least one of an angulation (with respect to a surface at the surgical site 32), a diameter, and a depth associated with the patient-specific EMS procedure at the surgical site 32. While not limiting, it may be advantageous, for example, for a length of the port 26 (illustrated as line “l” in FIG. 2) to be at least about 7 mm to stabilize the trephine bur 34 during the procedure.

The surgical guide 12 may be constructed from a variety of materials, including, for example, any material that is sufficiently non-compliant so as to maintain a structure corresponding to the patient's dentition but yet is nonabrasive so as to not damage the patient's teeth. Such materials may include, for example, resin, polyether, polyvinyl siloxane, and vinyl polyether siloxane. For purposes of speed and simplicity, the surgical guide 12 may be fabricated by 3D printing, the filament for which may comprise any suitable, conventional material known to those of ordinary skill in the art having the benefit of the disclosure made herein.

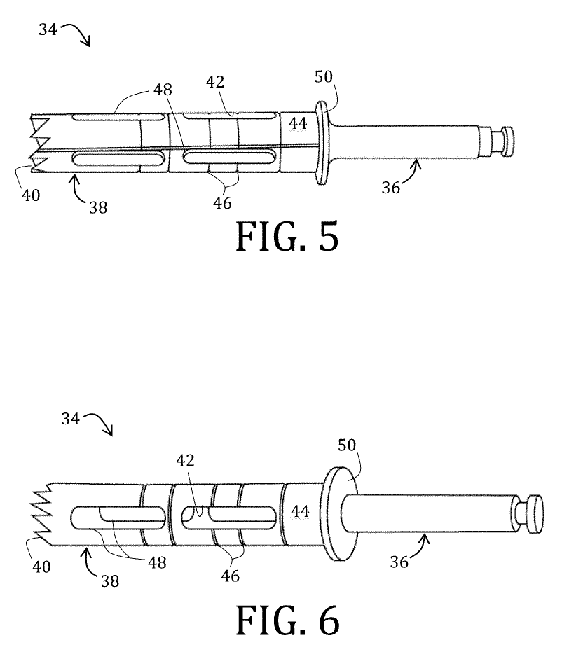

With reference now to FIGS. 4-6, the trephine bur 34 according to an embodiment of the present invention includes a proximally positioned shank 36 and a distally positioned barrel 38 having a bur edge 40 constructed from a non-reactive, surgical material, such as titanium or stainless steel. The barrel 38 has a bore 42 extending therethrough for receiving excised bone material. An exterior surface 44 of the barrel 38 may include a plurality of markings 46 to indicate depth of the bur edge 40. The markings 46 may be pigmented or otherwise drawn lines or grooves machined into the exterior surface 44. A plurality of openings 48 may extend between the exterior surface 44 to the bore 42 for providing visualization during the procedure and ease of bone removal from the bore 42 after the procedure.

The trephine bur 34 of FIGS. 4-6 further includes a stop 50 between the barrel 38 and the bore 42 that is configured to meet a proximal surface 52 (FIG. 2) of the port 26 (FIG. 2) and thereby create a positive stop such that the trephine bur 34 does not exceed a maximum cut depth. In this regard, a length of the barrel 38 may determine a maximum drill depth of the surgical procedure. As specifically illustrated in this embodiment, the stop 48 may be constructed from the same material as the barrel 38 and the shank 36 so as to form a unitary structure; however other structures and embodiments are possible, some of which are described in greater detail below.

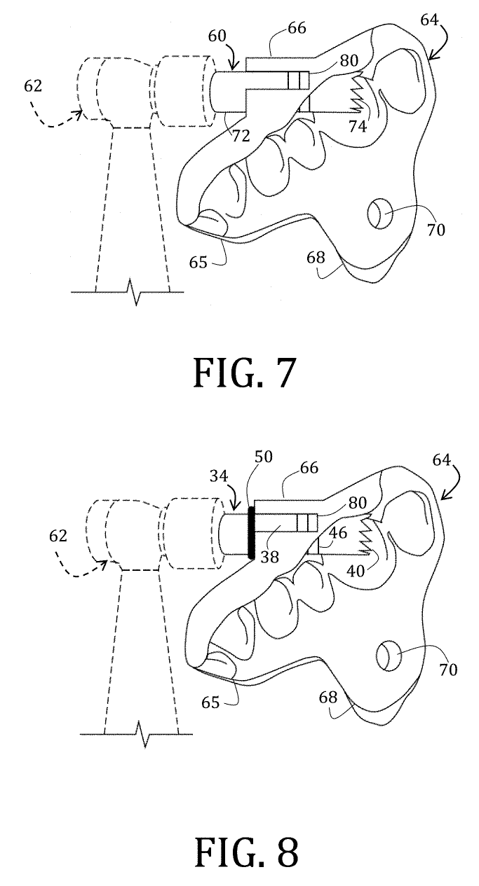

And so, with reference now to FIGS. 7 and 8, and continued reference to FIGS. 4-6, in FIG. 7 a conventional trephine bur 60 is used with a drill 62 and a surgical guide 64 (having a dentate guard 65, a first port 66 through which the trephine bur 60 extends, and a second port 68, with bore 70, not utilized in the present illustration) according to another embodiment of the present invention. The conventional trephine bur 60 incudes a barrel 72, a shank (not shown in FIG. 7), and a burred distal edge 74 of the barrel 70. FIG. 8 is similar but utilizes the trephine bur 34 of FIG. 4. As is shown, the stop 50 may be positioned adjacent to a proximal surface 76 of the first port 66 such that the trephine bur cannot further advance into the first port 66 (or the patient). A surgeon using the conventional trephine bur 60 would need to practice additional case to ensure a desired maximum depth is not exceeded. Thus embodiments of the trephine bur 34 provide a measure of safety and predictability not available with conventional dental trephine burs 60.

The particular embodiment of the surgical guide 64 illustrated in FIGS. 7 and 8 includes a side window 80 in the first port 66 that may be suitable for irrigation or insertion of other instrumentation. For example, irrigation or liquid coolant to be delivered to the trephine rotating within the port.

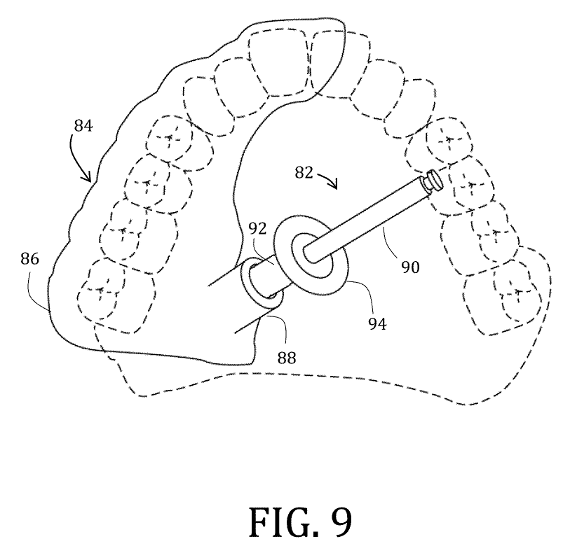

Referring now to FIG. 9, a trephine bur 82 in accordance with another embodiment of the present invention is shown in use with a surgical guide 84 (having a dentate guard 86 and palatally-positioned port 88) that is in accordance with another embodiment of the present invention. The trephine bur 82 includes a proximally positioned shank 90 and a distally positioned barrel 92 having a bur edge (not shown in FIG. 9) constructed from a non-reactive, surgical material, such as titanium or stainless steel. The barrel has a bore (not shown in FIG. 9) extending therethrough for receiving excised bone material. Although not shown in FIG. 9, but as was noted above, an exterior surface of the barrel may include a plurality of markings to indicate depth of the bur edge. Additionally although again not shown in FIG. 9, a plurality of openings may extend between the exterior surface to the bore for providing visualization during the procedure and ease of bone removal from the bore after the procedure.

The particular embodiment of FIG. 9 includes an adjustable stop 94, which may comprise a disk or washer surrounding the barrel 92 of the trephine bur 82 and which may configured to engage the proximal edge of the trephine bur port and prevent further advancing of the trephine bur. The stop 94 may be welded, friction fit, or otherwise affix or secured into a particular position on the barrel to define operational depth.

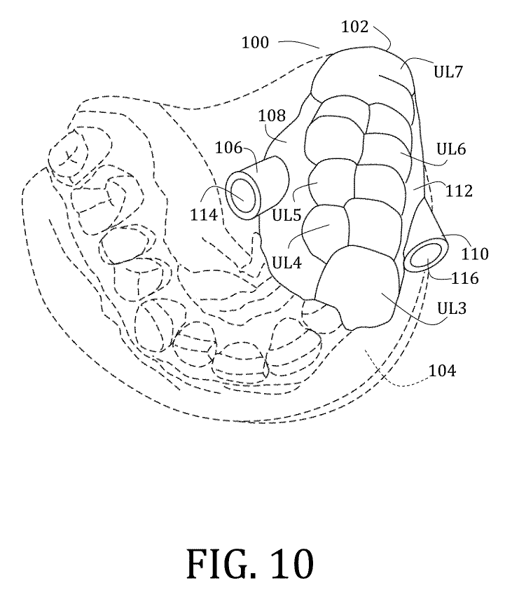

Turning now to FIG. 10, a surgical guide 100 in accordance with still another embodiment of the present invention is shown and includes a dentate guard 102 configured to conform to a patient's dental anatomy so as to stabilize the surgical guide 100 during the EMS procedure. The dentate guard 100, as shown, conforms to the left canine (UL3), bicuspids (UL4, UL5), the molars (UL6, UL7) and associated gingiva 104.

The surgical guide 100 further includes a first surgical port 106 on a palatal side 108 of the surgical guide 100 and a second surgical port 110 on the facial side 112 of the surgical guide 100. Each port 106, 110 includes a bore 114, 116 extending therethrough such that distal ends (not shown in FIG. 10) of each port 106, 110 aligns to respective first and second surgical sites (not shown in FIG. 10) for the patient. Diameters of each bore 114, 116 may be similar or different and may be configured to receive an appropriate trephine bur or other suitable instrument. As was noted above, the trephine bur size may be determined by such factors as root-end width, adjacent anatomical structures, considerations for visualization, surgeon's preference, or combinations thereof.

Each port 114, 116 may be positioned and dimensioned, with respect to the dentate guard, so as to specify at least one of an angulation (with respect to a surface at the surgical sites), a diameter, and a depth associated with the patient-specific EMS procedure at the surgical site. Lengths of each port 114, 116 may also vary but is not required. The surgical guide 100 may be constructed from, and using methods, that were described in detail above.

In use, and referring again to FIGS. 2-4, the surgical procedure may begin with optional soft tissue retraction. In that way, retractors may be used or, while not specifically illustrated herein but referenced in Example 3 below, the surgical guide 12 may include a seldin, a weider, or other similar structure extending from the dentate guard 22. In that regard, the retractor may be incorporated into the 3D model and fabricated with the surgical guide 12 as a unitary structure or may, otherwise, be retroactively affixed to the surgical guide 12.

After optional soft tissue retraction, the surgical guide 12 may be positioned onto the dentition (UL1, UR1, UR2, UR3) of the patient and secured thereto by friction fit to the particular geometry and anatomy of the patient.

The trephine bur 34 may then be advanced into the port 26 so as to engage the surgical site 32. If desired, the bur edge 40 of the trephine port 34 may be pressed into the tissue at the surgical site 32 so as to create bleeding points to delineate a mucosal window. Optionally an incision may be made proximate to the bleeding points. The trephine bur 32 may be rotated, with or without sterile water irrigation, to incrementally cut through the bone, root end, soft tissue, and so forth. If a side windows 80 (FIG. 7) had been included in the surgical guide design, then irrigation, visualization, or other tools may engage the surgical site 32 through the side window 80 (FIG. 7).

When the trephine osteotomy is complete, the trephine bur 32 may be retracted from the port 26 and the surgical guide 12 removed from the patient's teeth (UL1, UR1 UR2, UR3). If the cylindrical core of bone generated by the osteotomy remained in the surgical site 32, then the surgeon may remove the core as would be conventional. Otherwise, the core may be removed from within the barrel 38 of the trephine bur 34. The cylindrical core may include, in some instances, a root end, infected tissue, or combination thereof. One or more of these may be used for pathological assessment, if needed or desire. Additionally or alternatively, bone comprising the core may be used as an autogenous graft.

With the core removed, the resected root end and other structures within the surgical site 32 may be visualized. Certain tools may assist in visualization, such as a micro-mirror. The root end, surrounding bone, surgical site, and surrounding areas may be debrided and filled with a biocompatible material. Sutures may be used, in necessary, to close the surgical site 32 and/or any other incisions

The following examples illustrate particular properties and advantages of some of the embodiments of the present invention. Furthermore, these are examples of reduction to practice of the present invention and confirmation that the principles described in the present invention are therefore valid but should not be construed as in any way limiting the scope of the invention

### EXAMPLES

### Example 1—Surgical Preparation and Planning

For cases presented herein, unless otherwise specified, an 80 mm×80 mm preoperative cone beam computer tomography (“CBCT”) scan was carried out using a 3-D Accuitomo 170 (J Morita USA, Inc., Irvine, Calif.). Polyvinyl siloxane (“PVS”) impressions (Aquasil Ultra; Dentsply Caulk, Milford, Del.) were made and poured. To overcome restoration-associated artifacts, a digital 3D scan of the poured model, or in 1 case with minimally restored dentition, a CBCT scan of the PVS impression, was made and merged with the preoperative DICOM files. Care was taken to capture the alveolus at the surgical site during impression.

The cast was imaged by a 3 Shape D1000 benchtop scanner (Whip Mix Corp, Louisville, Ky.). The digital impression file was merged with the CBCT DICOM file in Mimics implant planning software (Materialise, Leuven, Belgium) or Blue Sky Plan 3 implant planning software (Blue Sky Bio, LLC, Grayslake, Ill.) for the design of the surgical guide.

Each surgical guide was designed with a port configured to accommodate a BIOMET 3i trephine bur (Palm Beach Gardens, Fla.) with the diameter, depth of penetration, angulation, and the site of root resection designed. Guide ports had a minimum depth of 7 mm to ensure trephine bur stabilization as determined during in vitro testing. The trephine bur diameter was selected based on root-end width, adjacent anatomic structures, and requirements for visualization. An irrigation window was created in the guide port to permit direct access for copious sterile saline for lubrication and cooling.

A stereolithography file of the surgical guide was produced and exported to a 3D printer (Objet 260 Connex3; Stratasys Ltd, Austin, Tex.). The 3D surgical guide was printed and an intimate fit verified with the poured cast.

After soft tissue reflection, the precise fit of the 3D surgical guide was verified. Two retractors cleared soft tissue from the surgical site. The trephine bur port provided protection to the soft tissue. A 5 mm or 6 mm outer-diameter hollow trephine bur was rotated at 1200 rpm with maximum torque in an electric hand piece (Anthogyr SAS, Sallanches, France) with sterile water irrigation, incrementally cutting through the bone, root end, and soft tissue with a light pecking motion over a period of time ranging from 1 minute to 2 minutes, depending on the depth of insertion.

After cutting, the trephine bur, the cylindrical core of bone, root end, and soft tissue were removed. The core specimen was submitted for biopsy.

Cases were completed under a surgical operating microscope (OPMI ProErgo; Zeiss Inc., Thornwood, N.Y.) to include ultrasonic root-end preparation and root-end filling with Endosequence BC Root Repair Material (Brasseler USA, Savannah, Ga.). Tissue was reapproximated and sutured.

For root-end resection of each case, respectively, adequate depth of penetration was designed and determined when the proximal extent of the trephine bur cylinder was flush with the orifice of the guide port (Example 2); a depth-defining washer was designed, printed, and placed over the shaft of the trephine bur such that the washer pressed against the guide port limiting penetration depth (Example 3); and the hand piece head touched the guide port (Example 4). Trephine burs with side venting, constant copious irrigation, and a gentle pecking motion allowed for osteotomy without excessive heat generation.

### Example 2—Maxillary Second Molar Palatal Root

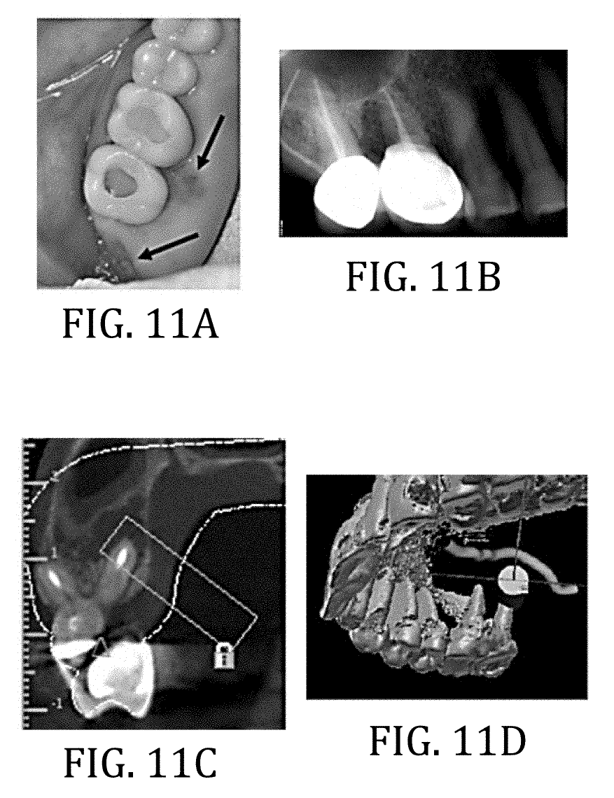

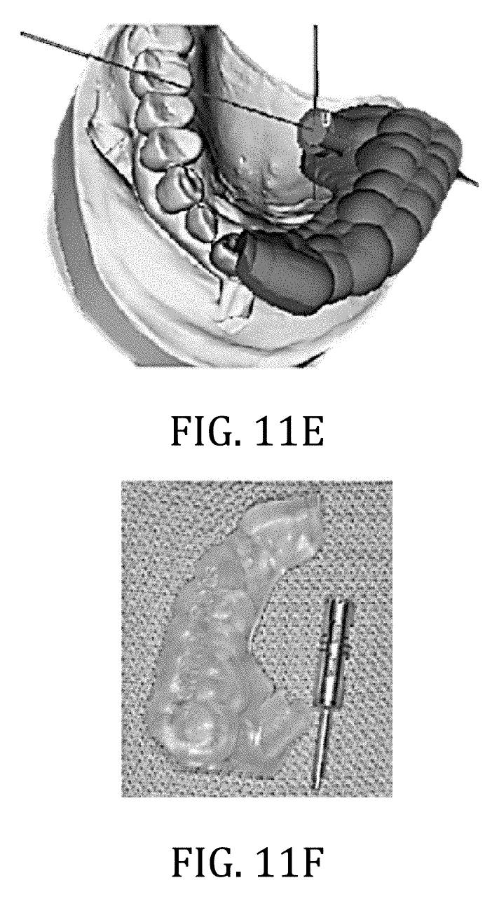

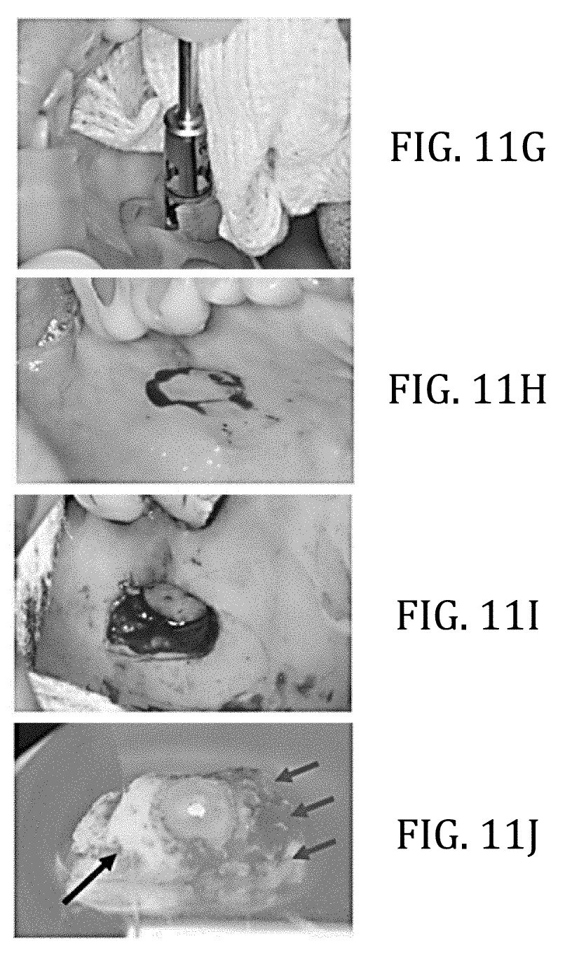

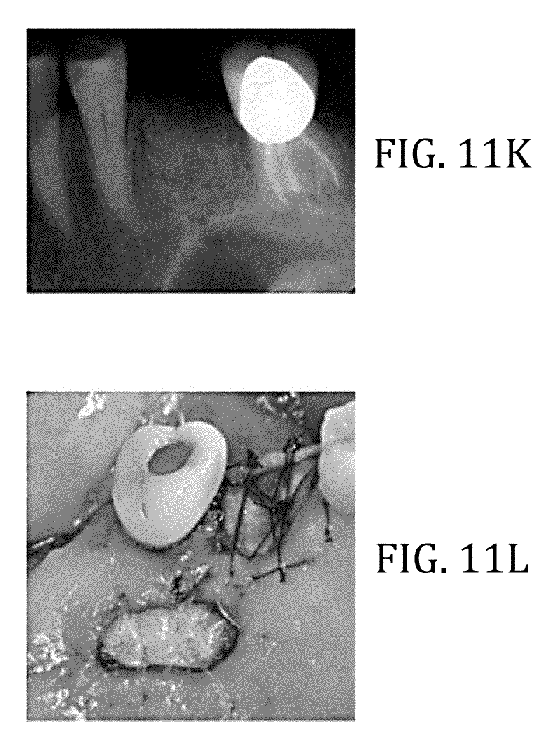

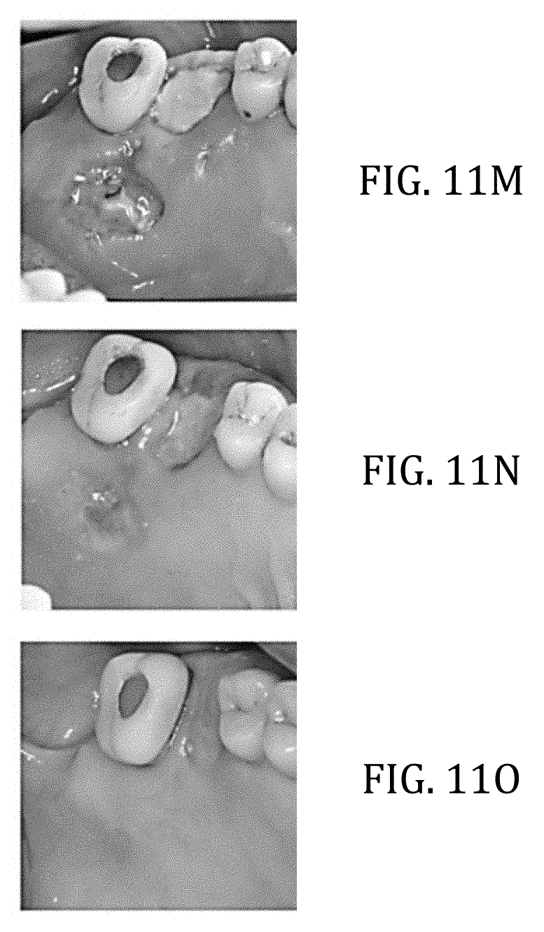

A 66-year-old American Society of Anesthesiologists (ASA) class I woman taking no medications presented with biting pain in the posterior maxilla. Approximately 1 year before evaluation, tooth #1 was extracted, tooth #2 received nonsurgical root canal treatment, and tooth #3 received retreatment for a long-standing perforation and missed second mesiofacial canal. Tooth #3 had a 9 mm probing depth at the mesiolingual and a 6 mm probing depth at the distolingual. Two sinus tracts are identified in FIG. 11A with black arrows: a first sinus tract was present at the base of the lingual papilla between tooth #2 and tooth #3 and traced radiographically to the palatal root of tooth #3; and a second sinus tract was present overlying alveolar bone 4 mm posterior to the distal marginal ridge of tooth #2 and traced to the palatal root of tooth #2 (see FIG. 11B).

For tooth #2, CBCT imaging revealed a 7 mm×5 mm×5 mm low density area at the apex of the palatal root with osseous healing at the mesiofacial and distofacial root ends compared with images from 1 year earlier. For tooth #3, CBCT imaging revealed an 8 mm×8 mm×6 mm low density area at the apex of the palatal root extending into the furcation, indicating failure of an attempted perforation repair with a hopeless prognosis. Tooth #2 diagnosis was previously treated with a chronic apical abscess, and the patient elected to have palatal root-end surgery in conjunction with extraction and ridge preservation of tooth #3.

The surgical site in this example was near the palatine artery, and surgery by traditional EMS would have been unacceptably risky. A surgical guide according to embodiments of the present invention with the port and bore configured to precisely direct a trephine bur to the surgical site (thus avoiding the palatine artery), permitted safe and effective EMS to be performed on this patient. Specifically, the port and bore were dimensioned so as to receive a 6 mm outer diameter trephine bur oriented to accommodate a palatal approach with clearance of the occlusal table on the contralateral side. Design of the surgical guide was such that the greater palatine artery was preserved, the palatal root was complete resected, and perforation of a pneumatized maxillary sinus between the facial and palatal roots was avoided.

FIG. 11C is a CBCT, coronal view of a planned trephine path while FIG. 11D is 3D model view of the planned trephine path that is positioned to avoid the GPA traced in yellow from the greater palatine foramen running anteriorly. FIG. 11E shows the 3D model of the surgical guide on a digital cast while FIG. 11F is a photograph of the surgical guide with a custom trephine bur.

The surgical guide was positioned onto the patient, and the trephine bur was directed through the port and pressed against the mucosa to create bleeding points (FIG. 11G) to define borders of a full-thickness mucosal “window” excision at the site of osteotomy (FIG. 11H). A full-thickness 8 mm×6 mm window of palatal tissue was excised and placed in Hank's Balanced Salt Solution (Lonza, Walkersville, Md.). Targeted EMS was performed, with FIG. 11I illustrating the mucosal window after trephine osteotomy with core in place. FIG. 11J is a photograph of the core specimen with palatal cortical bone (black arrow), resected root end, and soft tissue (blue arrows).

Root-end preparation and fill were accomplished. Bio-Oss (Geistlich Biomaterials, Princeton, N.J.) was placed in the osteotomy for tooth #2, and the palatal tissue was replaced and secured with 6-0 Monocryl Plus (Ethicon US LLC, Cornelia, Ga.). A freeze-dried bone allograft (Stryker, Kalamazoo, Mich.) with an Osseoguard membrane (BIOMET 3i) was placed and secured with 4-0 Monocryl sutures (Ethicon US LLC) at the extraction socket of tooth #3. Exemplary postoperative radiograph and photograph are shown in FIGS. 11K and 11L, respectively.

At 2 weeks, all sutures were removed. The excised palatal tissue was replaced by new mucosa at 6 weeks. A biopsy report for tooth #2 described a periapical cyst. The patient remained asymptomatic throughout a 12 week follow-up period.

Sequential photographs of healing at 2 weeks, 4 weeks, and 3 months are shown in FIGS. 11M-11O, respectively.

The surgical guide ensured the greater palatine artery, which coursed near the surgical site, was preserved. The palatal tissue was replaced over the surgical site for patient comfort during anticipated healing by secondary intention. It is uncertain whether peripheral areas of the replanted tissue remained vital surrounding a central area of necrosis or if healing was completely by secondary intention.

### Example 3—Maxillary First Molar Fused DF-Palatal Root

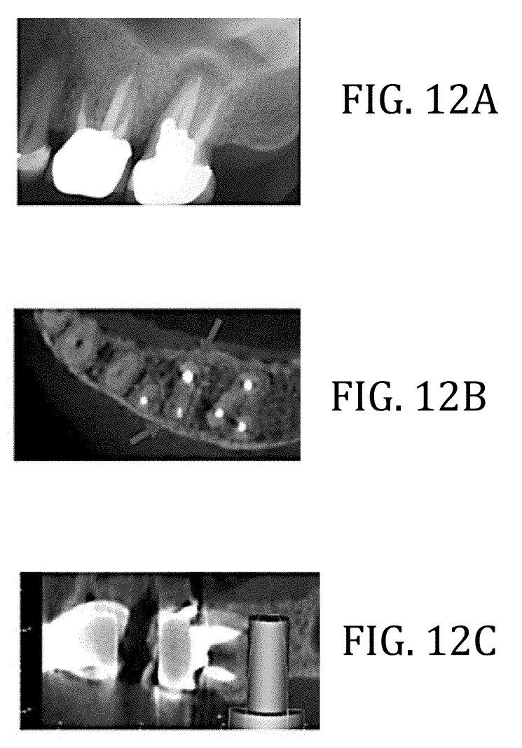

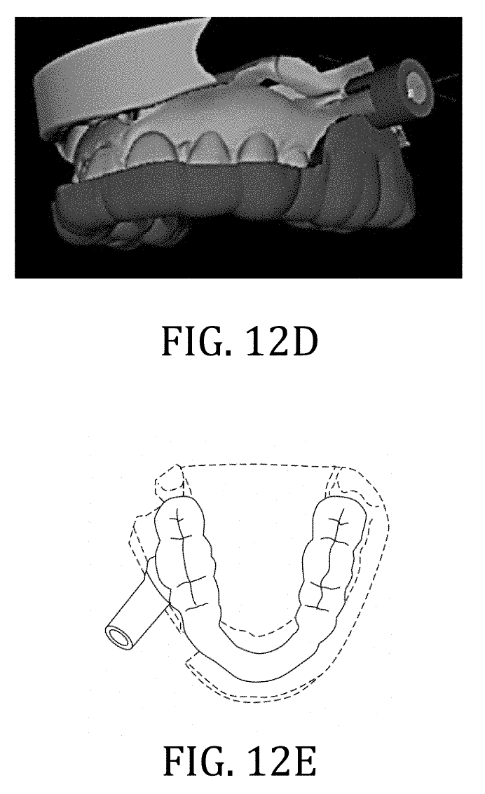

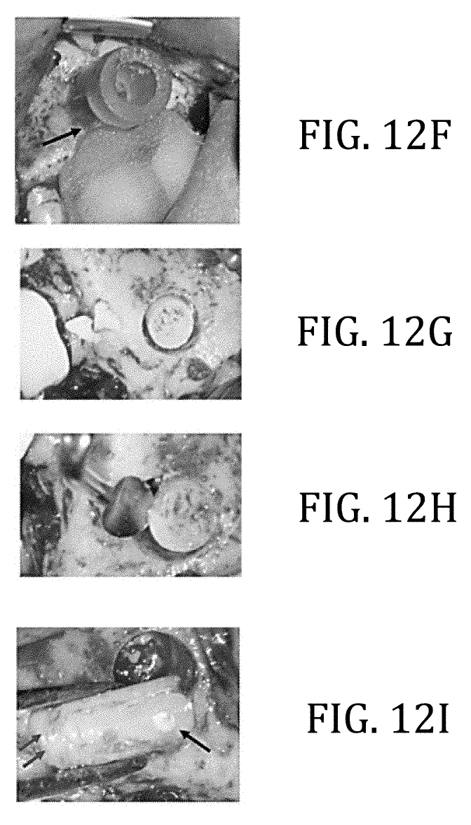

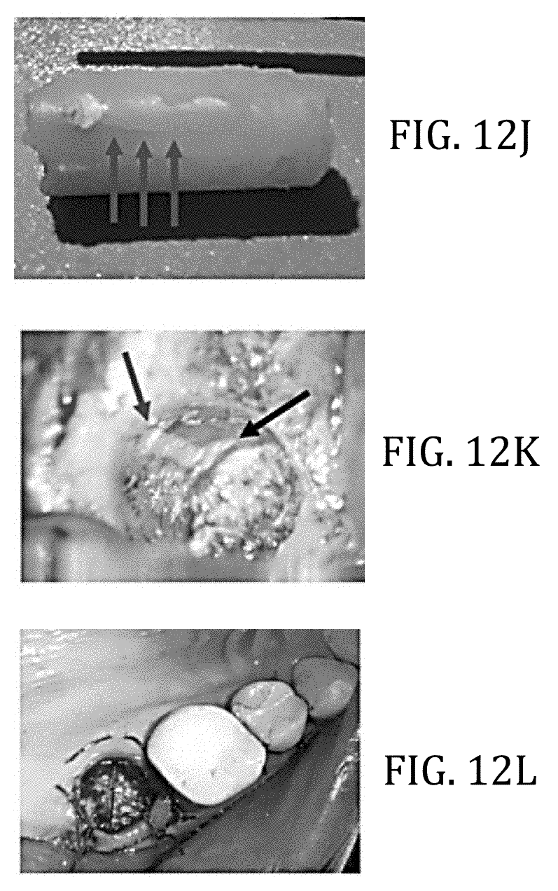

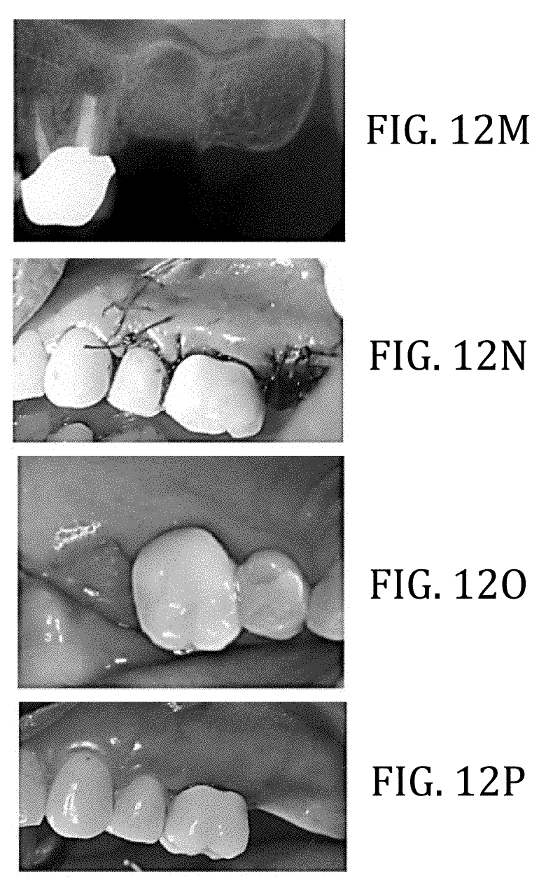

A 39-year-old ASA class 1 woman taking no medications presented with left maxillary posterior biting pain of several months duration. A preoperative radiograph is shown in FIG. 12A. Tooth #14 and tooth #15 received root canal treatment several years prior. Clinical examination revealed probings all 4 mm or less for teeth #s 11-15. Tooth #12 and tooth #13 had short cold responses, no percussion or palpation tenderness, and physiologic mobility. Tooth #14 and tooth #15 had no cold response, no percussion or palpation tenderness, and physiologic mobility. Tooth #15 had biting pain with Tooth Slooth (Patterson Dental Supply, Inc., St. Paul, Minn.) over all cusps, reproducing the patient's chief complaint.

This patient presented with a fused DF and palatal root (see FIG. 12B) and thus was not a candidate for surgery by conventional EMS procedures.

CBCT imaging and radiographic examination revealed root canal treatment of tooth #14 with 3 canals obturated with a 1 mm palatal root overfill and a 7 mm×2 mm×1 mm low density area associated with the apex of a fused DF and palatal root (FIG. 12A). Tooth #15 had little coronal tooth structure remaining with prior mesial perforation of the MF root and an 8 mm×8 mm×6 mm low density area or “halo” radiolucency extending coronally on the mesial to a site of previous perforation near the osseous crest. Diagnosis for tooth #14 was previously treated with asymptomatic apical periodontitis and, after discussion of treatment alternatives, the patient elected to have apical surgery addressing the fused DF and palatal root. Diagnosis for tooth #15 was previously treated with symptomatic apical periodontitis, likely vertical root fracture, with a hopeless long-term prognosis, and the patient elected to receive extraction.

A PVS impression of the maxillary arch was made. FIG. 12C is a CBCT, coronal view of a planned trephine path. FIG. 12D shows the 3D model of the surgical guide on a digital cast while FIG. 12E is a diagrammatic view of the surgical guide, which included a port dimensioned to accommodate a 5 mm outer-diameter trephine bur at the angulation required to remove the fused DF-palatal root end with an insertion depth of 11 mm from the facial cortical plate.

The surgery was performed under intravenous sedation. A full-thickness mucoperiosteal flap was elevated, and the surgical guide was inserted (FIG. 12F). Targeted EMS was performed with FIG. 12G illustrating the mucosal window after trephine osteotomy with core in place. FIG. 12H illustrates removal of the core and FIG. 12I is a photograph of the core specimen revealing facial cortical bone (blue arrows), fused DF-palatal root end with palatal canal GP (black arrow). FIG. 12J is a photograph of the core with undebrided DF canal and isthmus (red arrows).

Retrograde fill of DF canal (blue arrow), palatal canal (black arrow), and a previously undebrided 6 mm isthmus was accomplished and are shown in the photograph of FIG. 12K. Tooth #15 was extracted, and Bio-Oss Collagen (Geistlich Biomaterials, Princeton, N.J.) was placed within the socket and the osteotomy before suturing with 4-0 Vicryl (Ethicon US LLC) and 4-0 Chromic Gut sutures (Ethicon US LLC) (see FIG. 12L). A biopsy report described a periapical cyst. The patient was asymptomatic at 1 week and 1 month.

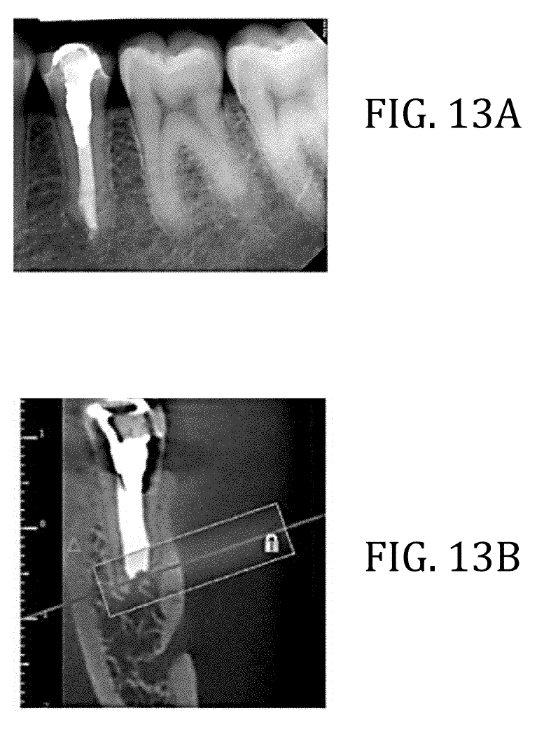

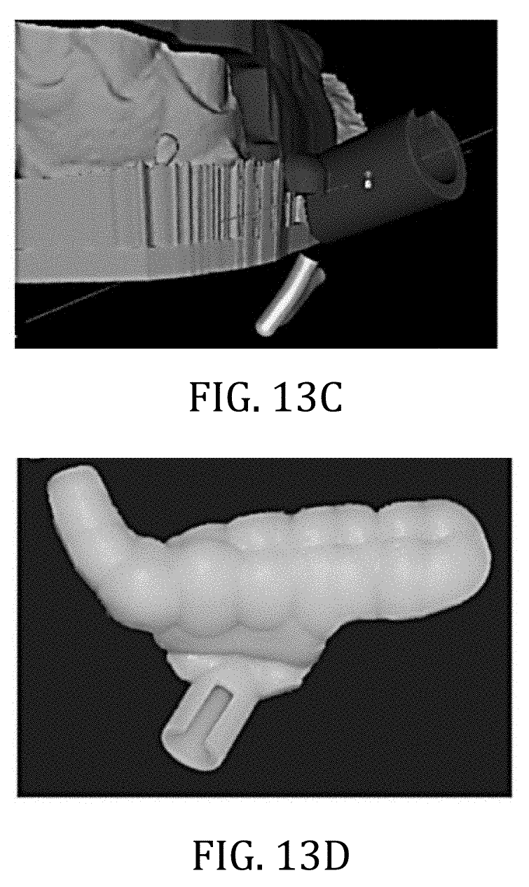

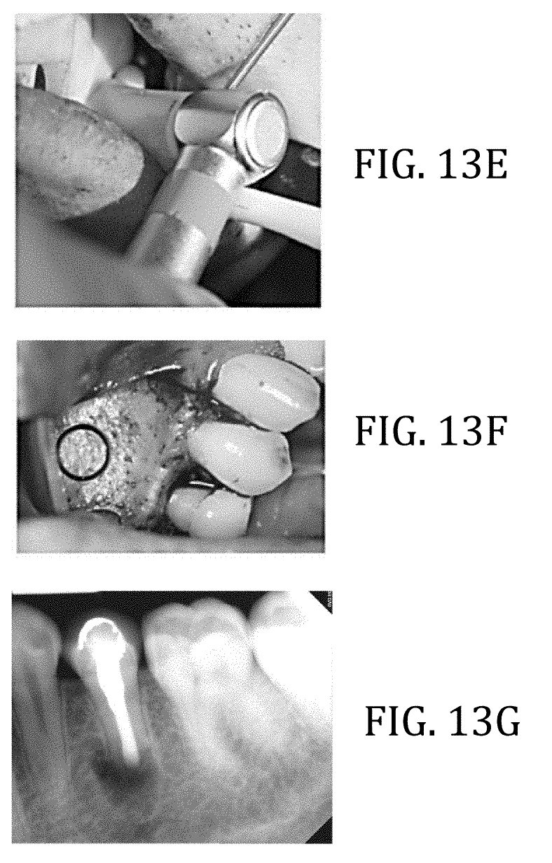

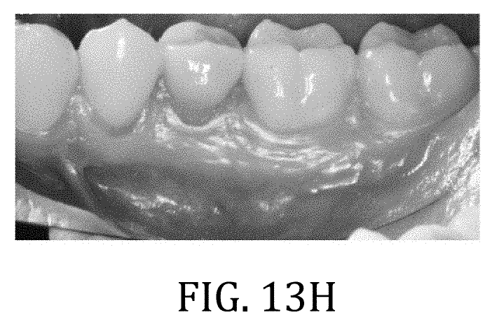

Exemplary postoperative radiograph and photograph are shown in FIGS. 12M and 12N, respectively. FIGS. 120 and 12P are 1 month postoperative photographs.

The surgical guide enabled an accurate 11 mm osteotomy depth for resection of the fused DF palatal root.

### Example 4—Mandibular Second Premolar

A 23-year-old ASA class I man taking no medications presented with pain upon biting in the mandibular left posterior. Eleven years prior, tooth #20 received immediate apexification treating pulp necrosis associated with dens evaginatus. Teeth #s 18-21 all had probings less than 3 mm. Teeth #s 18, 19, and 21 were unrestored and had short cold responses, no percussion or palpation tenderness, and physiologic mobility. Tooth #20 had a porcelain crown with adequate margins, no cold response, moderate percussion tenderness, no palpation tenderness, and physiologic mobility.

Radiographic (FIG. 13A) and CBCT imaging revealed a 2 mm×2 mm×2 mm low density area at the apex of tooth #20, which was 2.0 mm superior to the mental foramen. Diagnosis for tooth #20 was previously treated with symptomatic apical periodontitis, and the patient elected to have targeted EMS.

A PVS impression was imaged with a CBCT system, and resultant files were merged with preoperative CBCT imaging. The surgical guide was designed with the trephine port in a posture that would avoid trauma to the mental nerve. FIG. 13B is a CBCT, coronal view of a planned trephine path 1.5 mm from mental nerve exit. FIG. 13D shows the 3D model of the surgical guide on a digital cast, and FIG. 13D is a photograph of the surgical guide and included an irrigation window within the port.

The surgery was performed under oral sedation. A full-thickness mucoperiosteal flap was reflected without visualization of the mental nerve. Targeted EMS was performed. The surgical guide, once inserted, providing lip retraction (FIG. 13E). Osteotomy termination was reached when the handpiece touched the guide port.

Root-end inspection revealed serviceable white material consistent with tricalcium silicate cement, and no further root-end manipulation was conducted. Bone, root end, and soft tissue core prior to elevation are shown in FIG. 13F. The site was closed with 4-0 Vicryl and 5-0 Chromic Gut sutures. An exemplary postoperative radiograph is shown in FIG. 13G.

A biopsy report described a periapical granuloma.

At 1 week, the otherwise asymptomatic patient reported dysesthesia of the lower left lip to the midline for which he received a Medrol Dosepak (Pfizer, New York, N.Y.). At 1 month, the dysesthesia resolved, and the patient was asymptomatic. A two month postoperative photograph is shown in FIG. 13H.

The surgical guide ensured preservation of the mental nerve, which exited 2 mm apical to the trephine bur path.

According to embodiments of the present invention and as described herein, methods and surgical guides for successful surgical treatment in three anatomically challenging scenarios have been shown: (1) a palatal approach to the palatal root of a maxillary second molar, (2) a facial approach to a fused distofacial-palatal root of a maxillary first molar, and (3) a mandibular second premolar in close proximity to the mental foramen.

While the present invention has been illustrated by a description of one or more embodiments thereof and while these embodiments have been described in considerable detail, they are not intended to restrict or in any way limit the scope of the appended claims to such detail. Additional advantages and modifications will readily appear to those skilled in the art. The invention in its broader aspects is therefore not limited to the specific details, representative apparatus and method, and illustrative examples shown and described. Accordingly, departures may be made from such details without departing from the scope of the general inventive concept.

1. A surgical guide for endodontic micro-surgery (EMS) comprising:
a dentate guard configured to conformed to dentition of a patient proximate to a surgical site; and
a port extending from the dentate guard and having a bore extending therethrough such that a distal end of the bore terminates at the surgical site, the port and bore configured to receive a trephine bur for the EMS procedure at the surgical site.

2. The surgical guide of claim 1, wherein the port is positioned and dimensioned with respect to the dentate guard so as to specify at least one of an angulation, a diameter of the trephine bur, and an osteotomy depth of the EMS procedure.

3. The surgical guide of claim 1, wherein a diameter of the bore ranges from about 2.0 mm to about 8.0 mm.

4. The surgical guide of claim 1, wherein a length of the port ranges from about 4 mm to about 20 mm.

5. The surgical guide of claim 1, wherein the dentate guard and the port are a unitary structure comprising resin, polyether, polyvinyl siloxane, vinyl polyether siloxane, or combinations thereof.

6. The surgical guide of claim 1, wherein a proximal end of the port defines a maximum osteotomy depth.

7. The surgical guide of claim 1, wherein the port includes a side window configured for visualization, irrigation, instrument insertion, or combinations thereof.

8. The surgical guide of claim 1, further comprising:
a retractor extending from the dentate guard and configured to retract soft tissues from the surgical guide.

9. A method of performing a patient-specific endodontic micro-surgery (EMS) procedure, the method comprising:
positioning the surgical guide of claim 1 on the dentition of a patient; and
performing an osteotomy through the port.

10. The method of claim 9, wherein performing the osteotomy includes inserting a trephine bur through the port.

11. The method of claim 9, further comprising:
resecting a root end of a tooth.

12. A method for single-step osteotomy, root-end resection, and biopsy of a tooth, the method comprising:
positioning the surgical guide of claim 1 on the dentition of a patient;
inserting a trephine bur through the port of the surgical guide; and
advancing the trephine bur into the surgical site.

13. A surgical guide for endodontic micro-surgery (EMS) comprising:
a dentate guard configured to conform to dentition of a patient proximate to a first surgical site and a second surgical site;
a first port extending from the dentate guard and having a first bore extending therethrough such that a distal end of the first bore terminates at the first surgical site; and
a second port extending from the dentate guard and having a second bore extending therethrough such that a distal end of the second bore terminates at the second surgical site,
wherein each of the first and second ports and the respective first and second bores is configured to receive a trephine bur for the EMS procedure at the respective first and second surgical sites.

14. The surgical guide of claim 13, wherein each of the first and second ports is positioned and dimensioned with respect to the dentate guard so as to specify at least one of an angulation, a diameter of the trephine bur, and an osteotomy depth of the EMS procedure for the respective first and second surgical sites.

15. The surgical guide of claim 13, wherein a diameter of bore ranges from about 2.0 mm to about 8.0 mm.

16. The surgical guide of claim 13, wherein a length of each of the first and second ports independently ranges from about 4 mm to about 20 mm.

17. The surgical guide of claim 13, wherein the dentate guard and the first and second ports are a unitary structure comprising resin, polyether, polyvinyl siloxane, vinyl polyether siloxane, or combinations thereof.

18. The surgical guide of claim 13, wherein a proximal end of each of the first and second ports defines a maximum osteotomy depth for the respective first and second surgical sites.

19. The surgical guide of claim 13, wherein the first port, the second port, or both includes a side window configured for visualization, irrigation, instrument insertion, or combinations thereof.

20. The surgical guide of claim 13, further comprising:
a retractor extending from the dentate guard and configured to retract soft tissues from the surgical guide.

21. The surgical guide of claim 13, wherein the first port is on a palatal side of the dentate guard and the second port is on a facial side of the dentate guard.

22-27. (canceled)
